(19) United States
(12) Patent Application Publication
(10) Pub. No.: US 2003/0153054 A1
(43) Pub. Date: Aug. 14, 2003

(54) Methods and compositions for use in spliceosome mediated RNA trans-splicing

(76) Inventors: Lloyd G. Mitchell, Bethesda, MD (US); Mariano A. Garcia-Blanco, Durham, NC (US); Madaiah Puttaraju, Germantown, MD (US)

Correspondence Address: BAKER & BOTTS, 30 ROCKEFELLER PLAZA, NEW YORK, NY, 10112

(21) Appl. No.: 10/075,028

(22) Filed: Feb. 12, 2002

(51) Int. Cl. C12P 19/34; C12N 5/00

(52) U.S. Cl. 435/91.2; 435/375

(57) ABSTRACT

The present invention provides methods and compositions for delivery of synthetic pre-trans-splicing molecules (synthetic PTMs) into a target cell. The compositions of the invention include synthetic pre-trans-splicing molecules (PTMs) with enhanced stability against chemical and enzymatic degradation. The synthetic PTMs are designed to interact with a natural target precursor messenger RNA molecule (target pre-mRNA) and mediate a trans-splicing reaction resulting in the generation of a novel chimeric RNA molecule (chimeric RNA).

The present invention was made with government support under Grant Nos. SBIR R43DK56526-01 and SBIR5R44DK56526-03. The government has certain rights in the invention.

### 1. INTRODUCTION

The present invention provides methods and compositions for delivery of synthetic pre-trans-splicing molecules (synthetic PTMs) into a target cell. The compositions of the invention include synthetic pre-trans-splicing molecules (PTMs) with enhanced stability against chemical and enzymatic degradation. The synthetic PTMs are designed to interact with a natural target precursor messenger RNA molecule (target pre-mRNA) and mediate a trans-splicing reaction resulting in the generation of a novel chimeric RNA molecule (chimeric RNA). The PTMs of the invention are synthetically produced and transferred to a cell so as to result in the production of a novel chimeric RNA which may itself perform a function, such as inhibiting the translation of the RNA, or that encodes a protein that complements a defective or inactive protein in a cell, or encodes a toxin which kills specific cells, or that encodes a marker gene for imaging purposes. Generally, the target pre-mRNA is chosen as a target because it is expressed within a specific cell type thus providing a means for targeting expression of the novel chimeric RNA to a selected cell type. The methods of the invention encompass the synthetic production of PTMs, preferably in such a way as to possess enhanced stability against chemical and enzymatic degradation. The methods of the invention further comprise contacting the synthetic PTMs of the invention with a cell expressing a target pre-mRNA under conditions in which the synthetic PTM is taken up by the cell and a portion of the synthetic PTM is trans-spliced to a portion of the target pre-mRNA to form a novel chimeric RNA molecule. The methods and compositions of the invention can be used in cellular gene regulation, gene repair and suicide gene therapy for treatment of proliferative disorders such as cancer or treatment of genetic, autoimmune or infectious diseases. The present invention is based on the observation that direct delivery of in vitro synthesized synthetic PTMs into a target host cell can mediate accurate trans-splicing of target pre-mRNAs to generate novel chimeric RNAs. The present invention bypasses the requirement for efficient delivery of DNA molecules encoding a PTM into a target cell followed by expression of the DNA molecule to form a PTM capable of mediating a trans-splicing reaction.

### 2. BACKGROUND OF THE INVENTION

### 2.1 RNA Splicing

DNA sequences in the chromosome are transcribed into pre-mRNAs which contain coding regions (exons) and generally also contain intervening non-coding regions (introns). Introns are removed from pre-mRNAs in a precise process called splicing (Chow et al, 1977, Cell 12:1-8; and Berget, S. M. et al., 1977, Proc. Natl. Acad. Sci. USA 74:3171-3175). Splicing takes place as a coordinated interaction of several small nuclear ribonucleoprotein particles (snRNP's) and many protein factors that assemble to form an enzymatic complex known as the spliceosome (Moore et al, 1993, in The RNA World, R. F. Gestland and J. F. Atkins eds. (Cold Spring Harbor Laboratory Press, Cold Spring Harbor, N.Y.); Kramer, 1996, Annu. Rev. Biochem., 65:367-404; Staley and Guthrie, 1998, Cell 92:315-326).

Pre-mRNA splicing proceeds by a two-step mechanism. In the first step, the 5′ splice site is cleaved, resulting in a “free” 5′ exon and a lariat intermediate (Moore, M. J. and P. A. Sharp, 1993, Nature 365:364-368). In the second step, the 5′ exon is ligated to the 3′ exon with release of the intron as the lariat product. These steps are catalyzed in a complex of small nuclear ribonucleoproteins and proteins called the spliceosome. The splicing reaction sites are defined by consensus sequences around the 5′ and 3′ splice sites. The 5′ splice site consensus sequence is AG/GURAGU (where A=adenosine, U=uracil, G=guanine, C=cytosine, R=purine and /=the splice site). The 3′ splice region consists of three separate sequence elements: the branch point or branch site, a polypyrimidine tract and the 3′ splice consensus sequence (YAG). These elements loosely define a 3′ splice region, which may encompass 100 nucleotides of the intron upstream of the 3′ splice site. The branch point consensus sequence in mammals is YNYURAC (where N=any nucleotide, Y=pyrimidine). The underlined A is the site of branch formation (the BPA=branch point adenosine). The 3′ splice consensus sequence is YAG/G. Between the branch point and the splice site there is usually found a polypyrimidine tract, which is important in mammalian systems for efficient branch point utilization and 3′ splice site recognition (Roscigno, R., F. et al., 1993, J Biol. Chem. 268:11222-11229). The first YAG trinucleotide downstream from the branch point and polypyrimidine tract is the most commonly used 3′ splice site (Smith, C.W. et al., 1989, Nature 342:243-247).

In most cases, the splicing reaction occurs within the same pre-mnRNA molecule, which is termed cis-splicing. Splicing between two independently transcribed pre-mRNAs is termed trans-splicing. Trans-splicing was first discovered in trypanosomes (Sutton & Boothroyd, 1986, Cell 47:527; Murphy et al., 1986, Cell 47:517) and subsequently in nematodes (Krause & Hirsh, 1987, Cell 49:753); flatworms (Rajkovic et al., 1990, Proc. Nat'l. Acad. Sci. USA, 87:8879; Davis et al., 1995, J Biol. Chem. 270:21813) and in plant mitochondria (Malek et al., 1997, Proc. Nat'l. Acad. Sci. USA 94:553). In the parasite Trypanosoma brucei, all mRNAs acquire a splice leader (SL) RNA at their 5′ termini by trans-splicing. A 5′ leader sequence is also trans-spliced onto some genes in Caenorhabditis elegans. This mechanism is appropriate for adding a single common sequence to many different transcripts.

The mechanism of trans-splicing, which is nearly identical to that of conventional cis-splicing, proceeds via two phosphoryl transfer reactions. The first causes the formation of a 2′-5′ phosphodiester bond producing a ‘Y’ shaped branched intermediate, equivalent to the lariat intermediate in cis-splicing. The second reaction, exon ligation, proceeds as in conventional cis-splicing. In addition, sequences at the 3′ splice site and some of the snRNPs which catalyze the trans-splicing reaction, closely resemble their counterparts involved in cis-splicing.

Trans-splicing may also refer to a different process, where a portion of one pre-mRNA interacts with a portion of a second pre-mRNA, enhancing the recombination of splice sites between two conventional pre-mRNAs. This type of trans-splicing was postulated to account for transcripts encoding a human immunoglobulin variable region sequence linked to the endogenous constant region in a transgenic mouse (Shimizu et al., 1989, Proc. Nat'l. Acad. Sci. USA 86:8020). In addition, trans-splicing of c-myb pre-RNA has been demonstrated (Vellard, M. et al., Proc. Nat'l. Acad. Sci., 1992 89:2511-2515) and more recently, RNA transcripts from cloned SV40 trans-spliced to each other were detected in cultured cells and nuclear extracts (Eul et al., 1995, EMBO. J 14:3226). However, naturally occurring trans-splicing of mammalian pre-mRNAs is thought to be an exceedingly rare event.

In addition to splicing mechanisms involving the binding of multiple proteins to the precursor mRNA which then act to correctly cut and join RNA, a third mechanism involves cutting and joining of the RNA by the intron itself, by what are termed catalytic RNA molecules or ribozymes. The cleavage activity of ribozymes has been targeted to specific RNAs by engineering a discrete “hybridization” region into the ribozyme. Upon hybridization to the target RNA, the catalytic region of the ribozyme cleaves the target. It has been suggested that such ribozyme activity would be useful for the inactivation or cleavage of target RNA in vivo, such as for the treatment of human diseases characterized by production of foreign of aberrant RNA. The use of antisense RNA has also been proposed as an alternative mechanism for targeting and destruction of specific RNAs. In such instances small RNA molecules are designed to hybridize to the target RNA and by binding to the target RNA prevent translation of the target RNA or cause destruction of the RNA through activation of nucleases.

Until recently, the practical application of targeted trans-splicing to modify specific target genes has been limited to group I ribozyme-based mechanisms. Using the Tetrahymena group I ribozyme, targeted trans-splicing was demonstrated in E. coli (Sullenger B. A. and Cech. T. R., 1994, Nature 341:619-622), in mouse fibroblasts (Jones, J. T. et al., 1996, Nature Medicine 2:643-648), human fibroblasts (Phylacton, L. A. et al. Nature Genetics 18:378-381) and human erythroid precursors (Lan et al., 1998, Science 280:1593-1596). Several applications of targeted RNA trans-splicing driven by modified group I ribozymes have been explored. However, targeted trans-splicing mediated by native mammalian splicing machinery, i.e., spliceosomes, has only recently been reported.

U.S. Pat. Nos. 6,083,702, 6,013,487 and 6,280,978 describe the use of PTMs to mediate a trans-splicing reaction by contacting a target precursor MRNA to generate novel chimeric RNAs. The resulting RNA can encode any gene product including a protein of therapeutic value to the cell or host organism, a toxin, such as Diptheria, which causes killing of the specific cells or a novel protein not normally present in cells. The PTMs can also be engineered for the identification of exon/intron boundaries of pre-mRNA molecules using an exon tagging method and for production of chimeric proteins with peptide affinity purification tags which can be used to purify and identify proteins expressed in a specific cell type.

### 2.2 Nucleic Acid Transfer Into Cells

Such nuclear localization signals include small polypeptide sequnces that act as signals for translocation to the cell nucleus (Dingwall and Laskey, 1986, Ann. Rev. Cell Biol. 2:367-390; Dingwall and Laskey 1991, Trends Biochem. Sci. 16:478-481).

A variety of different methods are available for delivery of nucleic acid molecules into host cells. Such methods include direct injection of naked nucleic acid molecules, microparticle bombardment of nucleic acids (e.g., a gene gun; Bio-Rad, Dupont), coating nucleic acids with lipids or cell-surface receptors or transfecting agents and encapsulation of nucleic acids in liposomes, microparticles, or microcapsules. Other techniques of gene delivery include calcium phosphate mediated transfection, cationic polymer mediated transfection, lipofection or electroporation. Uncertainties relating to the mechanism of uptake into the cytoplasm and trafficking of DNA to the nucleus where transcription occurs complicate these methods and contribute to low transfection efficiencies. In addition, insufficient transcription of the transferred DNA can result in decreased levels of gene expression.

Though the use of attenuated viral vectors, such as adenovirus (Kozarsky and Wilson, 1993, Current Opinion in Genetics and Development 3:499-503), retrovirus (U.S. Pat. No. 4980286), herpes virus, and adeno-associated virus (AAV), achieve higher transfection efficiencies, viral vectors can be toxic and generate a host immune response. Retroviral infection can be carcinogenic and stimulate an immune response to viral proteins, causing local inflammation and posing a deterrent to repeat administration. Viral vectors, with the exceptions of AAV and lentivirus, are also limited because they can only infect replicating cells. Furthermore, these vectors must be designed with cell-specific, ubiquitous or inducible promoter systems. Accordingly, modulation of gene expression is an additional factor one must consider in the attempt to express a desired gene product in a cell.

The drawbacks associated with transfer of DNA into cells which must then be expressed can be overcome by the direct delivery of the RNA or chimeric (RNA/DNA/PNA, etc.) molecules to be expressed into the target host cell. The present invention bypasses the requirement for transfer of nucleic acid molecules capable of encoding a PTM into a target cell followed by efficient transcription of said molecules to form PTMs. The present invention is based on the successful delivery of synthetic PTMs into target cells and the demonstration that such synthetic molecules are capable of mediating trans-splicing reactions with target mRNAs to generate novel chimeric RNAs.

### 3. SUMMARY OF THE INVENTION

The present invention relates to compositions and methods of generating novel nucleic acid molecules through spliceosome-mediated targeted trans-splicing. Specifically, the invention provides methods and compositions for the delivery of synthetic PTMs into a target cell.

The compositions of the invention include synthetic pre-trans-splicing molecules (hereinafter referred to as “synthetic PTMs”) designed to interact with a natural target pre-mRNA molecule (hereinafter referred to as “pre-mRNA”) and mediate a spliceosomal trans-splicing reaction resulting in the generation of a novel chimeric RNA molecule (hereinafter referred to as “chimeric RNA”). The synthetic PTMs may comprise modified or substituted nucleotides which are preferred over naturally occurring nucleotides because of desirable properties such as, for example, increased, enhanced cellular uptake, increased targeting to the nucleus of the cell and/or enhanced binding to target cell or target pre-mRNA. In addition, carrier or excipients will be chosen based on their ability to stabilize the RNA molecules during in vitro formulation, their ability to increase the stability of the RNA in vivo and/or their ability to increase the efficiency of RNA transfer in vivo, thereby providing a more efficient RNA delivery system.

The methods of the invention encompass contacting the synthetic PTMs of the invention with a target cell expressing a natural target pre-mRNA under conditions in which the synthetic PTM is taken up by the cell and a portion of the synthetic PTM is spliced to the natural pre-mRNA to form a novel chimeric RNA. The synthetic PTMs of the invention are genetically engineered so that the novel chimeric RNA resulting from the trans-splicing reaction may itself perform a function such as inhibiting the translation of RNA, or alternatively, the chimeric RNA may encode a protein that complements a defective or inactive protein in the cell, or encodes a toxin which kills the specific cells. Generally, the target pre-mRNA is chosen because it is expressed within a specific cell type thereby providing a means for targeting expression of the novel chimeric RNA to a selected cell type. The target cells may include, but are not limited to those infected with viral or other infectious agents, benign or malignant neoplasms, or components of the immune system which are involved in autoimmune disease or tissue rejection. The PTMs of the invention may also be used to correct genetic mutations found to be associated with genetic diseases. In particular, double-trans-splicing reactions can be used to replace internal exons. The methods and compositions of the invention can be used in gene regulation, gene repair and targeted cell death. Such methods and compositions can be used for the treatment of various diseases including, but not limited to, genetic, infectious or autoimmune diseases and proliferative disorders such as cancer and to regulate gene expression in plants.

### 4. BRIEF DESCRIPTION OF THE DRAWINGS

FIG. 1A. Model of Pre-Trans-splicing RNA.

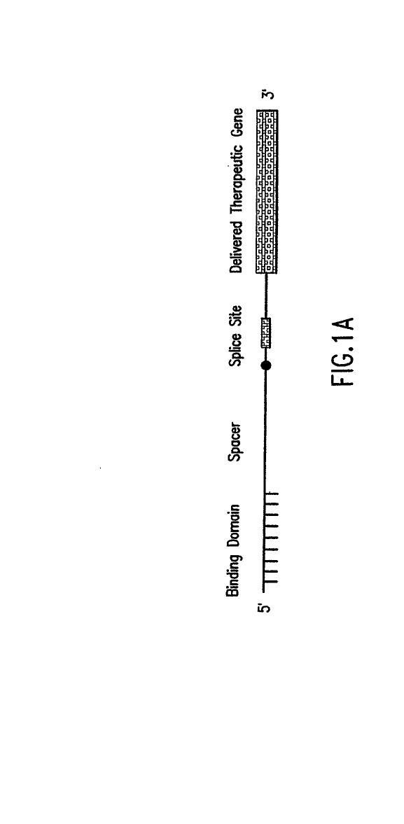

FIG. 1B. Model PTM constructs and targeted trans-splicing strategy. Schematic representation of linear PTMs (PTM+Sp and PTM−Sp). BD, binding domain; NBD, non-binding domain; BP, branch point; PPT, pyrimidine tract; ss, splice site and DT-A, diphtheria toxin subunit A. Unique restriction sites within the PTMS are indicated by single letters: E; EcoRI; X, Xhol; K, Kpnl; P, Pstl; A, Accl; B, BamHI and H; HindIII.

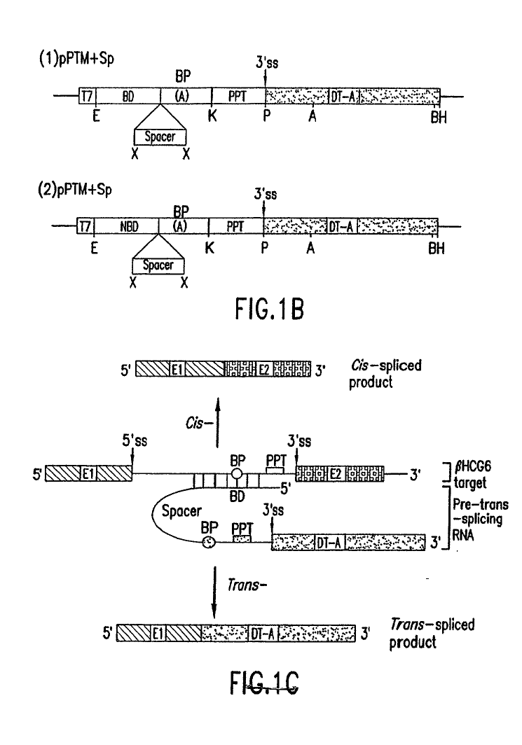

FIG. 1C. Schematic drawing showing the binding of PTM+Sp via conventional Watson Crick base pairing to the βHCG6 target pre-mRNA and the proposed cis- and trans-splicing mechanism.

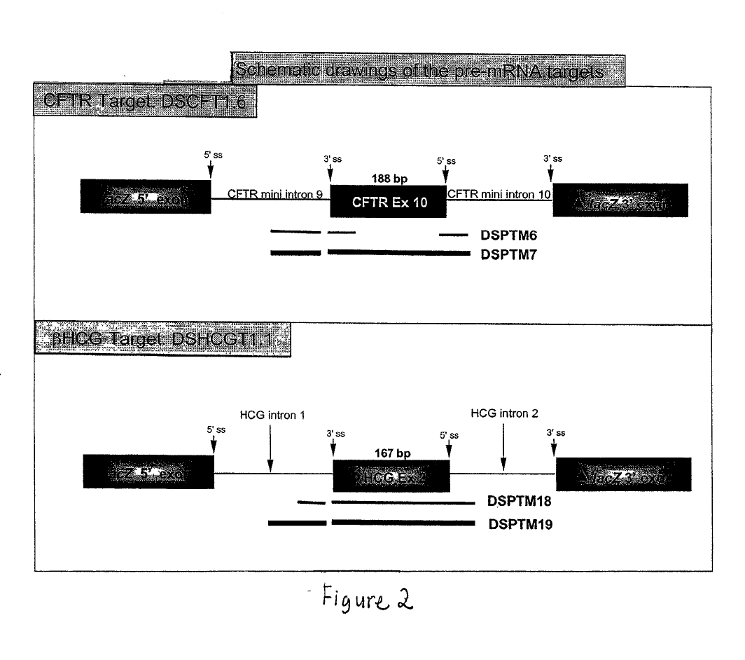

FIG. 2. Schematic drawings of constructed pre-mRNA targets (double trans-splicing).

FIG. 3. Schematic diagrams of double trans-splicing PTM.

FIG. 4. Diagram and important structural elements of double trans-splicing PTM7. The double splicing PTM7 has both 3′ and 5′ functional splice sites as well as binding domains.

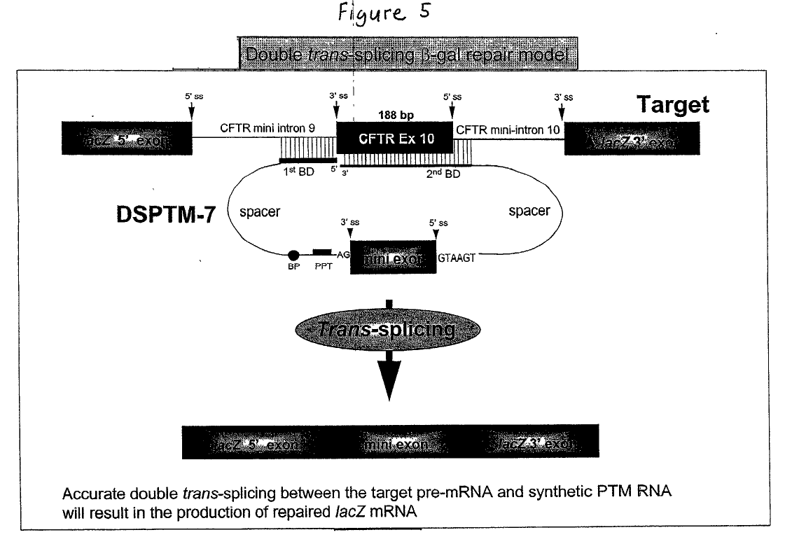

FIG. 5. Double trans-splicing β-gal repair model. Accurate double trans-splicing between the target pre-mRNA and synthetic PTM RNA will result in the production of repaired lacZ mRNA RNA.

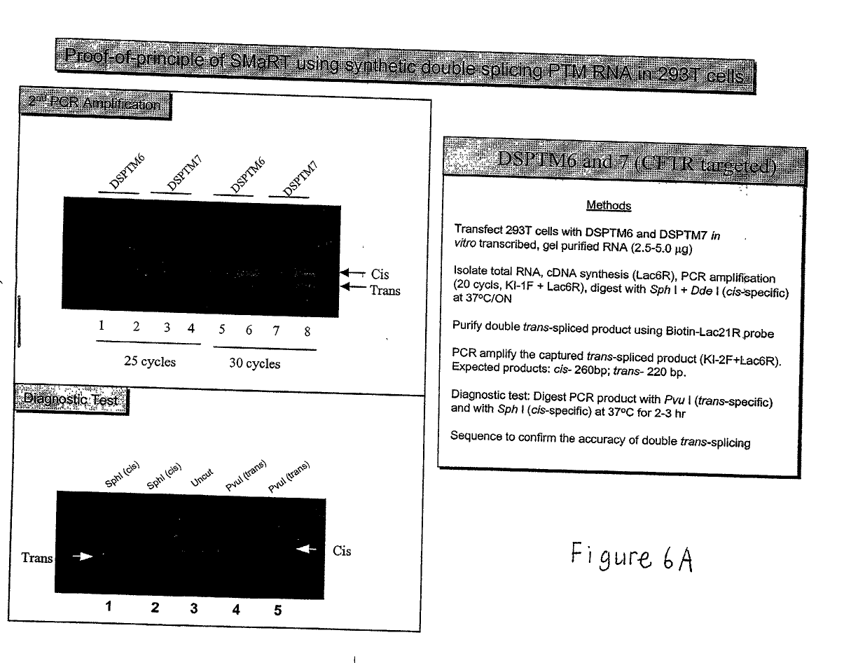

FIGS. 6A-B. Successful double trans-splicing of synthetic PTM RNA in transfected 293T cells.

FIG. 6C. The accuracy of double trans-splicing of synthetic PTM RNA in 293T cells was verified by sequencing the spliced RNA produced by RT-PCR.

FIG. 7. Restoration of β-gal function through RNA transfection in 293T cells.

### 5. DETAILED DESCRIPTION OF THE INVENTION

The present invention provides methods and compositions for delivery of synthetic PTMs into a target cell. The present invention relates to compositions comprising synthetic pre-trans-splicing molecules and a suitable carrier or incipient and the use of such compositions for generating novel nucleic acid molecules within a target cell. The synthetic PTMs are preferably produced with enhanced resistance to enzymatic and/or chemical degradation. In addition, the carriers or excipients are designed to stabilize the PTM during in vitro formulation, increase the stability of the PTM in vivo and/or increase the efficiency of PTM transfer in vivo, thereby providing a more efficient PTM delivery system.

The synthetic PTMs of the invention comprise one or more target binding domains that are designed to specifically bind to pre-mRNA, a 3′ splice region that includes a branch point, pyrimidine tract and a 3′ splice acceptor site and/or a 5′ splice donor site; and one or more spacer regions that separate the RNA splice site from the target binding domain. In addition, the synthetic PTMs of the invention can be engineered to contain any additional nucleotide sequences such as those encoding a translatable protein product.

The methods of the invention encompass contacting the synthetic PTMs of the invention with a target cell expressing a natural pre-mRNA under conditions in which a portion of the synthetic PTM is trans-spliced to a portion of the natural pre-mRNA to form a novel chimeric RNA. The target pre-mRNA is chosen as a target due to its expression within a specific cell type thus providing a mechanism for limiting expression of a novel RNA to a selected cell type. The resulting chimeric RNA may provide a desired function, or may produce a gene product in the specific cell type. The specific cells may include, but are not limited to those infected with viral or other infectious agents, benign or malignant neoplasms, or components of the immune system which are involved in autoimmune disease or tissue rejection. Specificity is achieved by modification of the binding domain of the PTM to bind to the target endogenous pre-mRNA. The gene products encoded by the chimeric RNA can be any gene, including genes having clinical applications, for example, therapeutic genes, marker genes and genes encoding toxins.

### 5.1 Structure of the Pre-trans-Splicing Molecules

The present invention provides compositions for use in generating novel chimeric nucleic acid molecules through targeted trans-splicing. The synthetic PTMs of the invention comprise (i) one or more target binding domains that targets binding of the synthetic PTM to a pre-mRNA (ii) a 3′ splice region that includes a branch point, pyrimidine tract and a 3′ splice acceptor site and/or 5′ splice donor site; and (iii) one or more spacer regions to separate the RNA splice site from the target binding domain. Additionally, the synthetic PTMs can be engineered to contain any nucleotide sequence encoding a translatable peptide or protein product. In yet another embodiment of the invention, the PTMs can be synthesized to contain nucleotide sequences that inhibit the translation of the chimeric RNA molecule. For example, the nucleotide sequences may contain translational stop codons or nucleotide sequences that form secondary structures and thereby inhibit translation. Alternatively, the chimeric RNA may function as an antisense molecule thereby inhibiting splicing, nuclear transport or translation of the RNA to which it binds.

A variety of different PTM molecules may be synthesized for use in the production of novel chimeric RNAs which may perform a function, such as inhibiting the translation of the RNA, or that encodes a protein that complements a defective or inactive protein in a cell or encodes a toxin which kills specific cells. Such PTMs include PTMs designed to correct defects in the cystic fibrosis gene or the clotting factor VIII gene, or those designed to inhibit viral gene products such as papilloma virus gene products. The design, construction and genetic engineering of such PTMs and demonstration of their ability to mediate successful trans-splicing reactions within the cell are described in detail in U.S. Pat. Nos. 6,083,702, 6,013,487 and 6,280,978 as well as patent Ser. No.09/941,492, each of which is incorporated by reference in their entirety herein.

The target binding domain of the synthetic PTM endow the synthetic PTM with a binding affinity. As used herein, a target binding domain is defined as any molecule, i.e., nucleotide, protein, chemical compound, etc., that confers specificity of binding and anchors the pre-mRNA closely in space to the synthetic PTM so that the spliceosome processing machinery of the nucleus can trans-splice a portion of the synthetic PTM to a portion of the pre-mRNA. The target binding domain of the synthetic PTM may contain multiple binding domains which are complementary to and in anti-sense orientation to the targeted region of the selected pre-mRNA. The target binding domains may comprise up to several thousand nucleotides. In preferred embodiments of the invention the binding domains may comprise at least 10 to 30 and up to several hundred or more nucleotides. The specificity of the synthetic PTM can be increased significantly by increasing the length of the target binding domain. For example, the target binding domain may comprise several hundred nucleotides or more. In addition, although the target binding domain may be “linear” it is understood that the RNA may fold to form secondary structures that may stabilize the complex thereby increasing the efficiency of splicing. A second target binding region may be placed at the 3′ end of the molecule and can be incorporated into the PTM of the invention. Absolute complementarily, although preferred, is not required. A sequence “complementary” to a portion of an RNA, as referred to herein, means a sequence having sufficient complementarity to be able to hybridize with the target pre-RNA, forming a stable duplex. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y). Generally, the longer the hybridizing nucleic acid, the more base mismatches with an RNA it may contain and still form a stable duplex. One skilled in the art can ascertain a tolerable degree of mismatch or length of duplex by use of standard procedures to determine the stability of the hybridized complex.

Where the PTMs are designed for use in intron-exon tagging or for peptide affinity tagging, a library of synthetic PTMs is genetically engineered to contain random nucleotide sequences in the target binding domain. Alternatively, for intron-exon tagging the synthetic PTMs may be genetically engineered so as to lack target binding domains. The goal of generating such a library of synthetic PTM molecules is that the library will contain a population of synthetic PTM molecules capable of binding to each RNA molecule expressed in the cell. A recombinant expression vector can be genetically engineered to contain a coding region for a PTM including a restriction endonuclease site that can be used for insertion of random DNA fragments into the PTM to form random target binding domains. The random nucleotide sequences to be included in the PTM as target binding domains can be generated using a variety of different methods well known to those of skill in the art, including but not limited to, partial digestion of DNA with restriction enzymes or mechanical shearing of DNA to generate random fragments of DNA. Random binding domain regions may also be generated by degenerate oligonucleotide synthesis. The degenerate oligonucleotides can be engineered to have restriction endonuclease recognition sites on each end to facilitate cloning into a PTM molecule for production of a library of PTM molecules having degenerate binding domains.

Binding may also be achieved through other mechanisms, for example, through triple helix formation, aptamer interactions, antibody interactions or protein/nucleic acid interactions such as those in which the PTM is engineered to recognize a specific RNA binding protein, i.e., a protein bound to a specific target pre-mRNA. Alternatively, the PTMs of the invention may be designed to recognize secondary structures, such as for example, hairpin structures resulting from intramolecular base pairing between nucleotides within an RNA molecule.

The PTM molecule also contains a 3′ splice region that includes a branch point, pyrimidine tract and a 3′ splice acceptor AG site and/or a 5′ splice donor site. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and /=the splice site). The 3′ splice site consists of three separate sequence elements: the branch point or branch site, a polypyrimidine tract and the 3′ splice site acceptor consensus sequence (YAG). The branch point consensus sequence in mammals is YNYURAC (Y=pyrimidine;N=any nucleotide). The underlined A is the site of branch formation. A polypyrimidine tract is located between the branch point and the splice site acceptor and is important for efficient branch point utilization and 3′ splice site recognition.

Further, PTMs comprising a 3′ acceptor site (AG) may be genetically engineered. Such PTMs may further comprise a pyrimidine tract and/or branch point sequence.

Recently, pre-messenger RNA introns beginning with the dinucleotide AU and ending with the dinucleotide AC have been identified and referred to as U12 introns. U12 intron sequences as well as any sequences that function as splice acceptor/donor sequences may also be used in PTMs.

A spacer region to separate the RNA splice site from the target binding domain may also included in the PTM. The spacer region can have features such as stop codons which would block any translation of an unspliced PTM and/or sequences that enhance trans-splicing to the target pre-mnRNA.

In a preferred embodiment of the invention, a “safety” is also incorporated into the spacer, binding domain, or elsewhere in the PTM to prevent non-specific trans-splicing. This is a region of the PTM that covers elements of the 3′ and/or 5′ splice site of the PTM by relatively weak complementarity, preventing non-specific trans-splicing. The PTM is designed in such a way that upon hybridization of the binding /targeting portion(s) of the PTM, the 3′ and/or 5′ splice site is uncovered and becomes fully active.

The “safety” consists of one or more complementary stretches of cis-sequence (or could be a second, separate, strand of nucleic acid) which weakly binds to one or both sides of the PTM branch point, pyrimidine tract, 3′ splice site and/or 5′ splice site (splicing elements), or could bind to parts of the splicing elements themselves. This “safety” binding prevents the splicing elements from being active (i.e. block U2 snRNP or other splicing factors from attaching to the PTM splice site recognition elements). The binding of the “safety” may be disrupted by the binding of the target binding region of the PTM to the target pre-mRNA, thus exposing and activating the PTM splicing elements (making them available to trans-splice into the target pre-mRNA).

A nucleotide sequence encoding a translatable protein capable of producing an effect, such as cell death, or alternatively, one that restores a missing function or acts as a marker, is included in the PTM of the invention. For example, the nucleotide sequence can include those sequences encoding gene products missing or altered in known genetic diseases. Alternatively, the nucleotide sequences can encode marker proteins or peptides which may be used to identify or image cells. In yet another embodiment of the invention nucleotide sequences encoding affinity tags such as, HIS tags (6 consecutive histidine residues) (Janknecht, et al., 1991, Proc. Natl. Acad. Sci. USA 88:8972-8976), the C-terminus of glutathione-S-transferase (GST) (Smith and Johnson, 1986, Proc. Natl. Acad. Sci. USA 83:8703-8707) (Pharmacia) or FLAG (Asp-Tyr-Lys-Asp-Asp-Asp-Asp-Lys) (Eastman Kodak/IBI, Rochester, N.Y.) can be included in PTM molecules for use in affinity purification. The use of PTMs containing such nucleotide sequences results in the production of a chimeric RNA encoding a fusion protein containing peptide sequences normally expressed in a cell linked to the peptide affinity tag. The affinity tag provides a method for the rapid purification and identification of peptide sequences expressed in the cell. In a preferred embodiment the nucleotide sequences may encode toxins or other proteins which provide some function which enhances the susceptibility of the cells to subsequent treatments, such as radiation or chemotherapy.

In a highly preferred embodiment of the invention a PTM molecule is fused to the Diphtheria toxin subunit A (Greenfield, L., et al., 1983, Proc. Nat'l. Acad. Sci. USA 80:6853-6857). Diphtheria toxin subunit A contains enzymatic toxin activity and will function if delivered into human cells resulting in cell death. Furthermore, various other known peptide toxins may be used in the present invention, including but not limited to, ricin, Pseudomonus toxin, Shiga toxin and exotoxin A.

Additional features can be added to the PTM molecule such as polyadenylation signals, or enhancer sequences to enhance splicing, additional binding regions, “safety”-self complementary regions, additional splice sites, or protective groups to modulate the stability of the molecule and prevent degradation. In an embodiment of the invention, sequences referred to as exonic splicing enhancers may also be included in the structure of the synthetic PTMs. Transacting splicing factors, namely the serine/arginine-rich (SR) proteins, have been shown to interact with such exonic splicing enhancers and modulate splicing (See, Tacke et al., 1999, Curr. Opin. Cell Biol. 11:358-362; Tian et al., 2001, J Biological Chemistry 276:33833-33839; Fu, 1995, RNA 1:663-680). Nuclear localization signals may also be included in the PTM molecule (Dingwell and Laskey, 1986, Ann Rev. Cell Biol. 2:367-390; Dingwell and Laskey, 1991, Trends in Biochem. Sci. 16:478-481). Such nuclear localization signals can be used to enhance the transport of synthetic PTMs into the nucleus where trans-splicing occurs. In addition, signals that enhance the retention of PTMs in the nucleus of the cell may be included in the PTM molecule.

Additional features that may be incorporated into the PTMs of the invention include stop codons or other elements in the region between the binding domain and the splice site to prevent unspliced pre-mRNA expression. In another embodiment of the invention, PTMs can be generated with a second anti-sense binding domain to promote binding to the 3′ target intron or exon and to block the fixed authentic cis-5′ splice site (U5 and/or U1 binding sites).

PTMs may also be generated that require a double-trans-splicing reaction for generation of a chimeric trans-spliced product. Such PTMs could be used to replace an internal exon which could be used for RNA repair. PTMs designed to promote two trans-splicing reactions are engineered as described above, however, they contain both 5′ donor sites and 3′ splice acceptor sites. In addition, the PTMs may comprise two or more binding domains and spacer regions. The spacer regions may be placed between the multiple binding domains and splice sites or alternatively between the multiple binding domains.

Further elements such as a 3′ hairpin structure, circularization RNA, ribonucleotide base modification, or a synthetic analog can be incorporated into PTMs to promote or facilitate nuclear localization and spliceosomal incorporation, and intracellular stability.

Additionally, when engineering PTMs for use in plant cells it may not be necessary to include conserved branch point sequences or polypyrimidine tracts as these sequences may not be essential for intron processing in plants. However, a 3′ splice acceptor site and/or 5′ splice donor site, such as those required for splicing in vertebrates and yeast, will be included. Further, the efficiency of splicing in plants may be increased by also including UA-rich intronic sequences. The skilled artisan will recognize that any sequences that are capable of mediating a trans-splicing reaction in plants may be used.

The PTMs of the invention can be modified at the base moiety, sugar moiety, or phosphate backbone, for example, to improve stability of the molecule, hybridization to the target mRNA, transport into the cell, etc. For example, modification of a PTM to reduce the overall charge can enhance the cellular uptake of the molecule. In addition modifications can be made to reduce susceptibility to nuclease or chemical degradation. The nucleic acid molecules may be synthesized in such a way as to be conjugated to another molecule such as a peptides (e.g., for targeting host cell receptors in vivo), or an agent facilitating transport across the cell membrane (see, e.g., Letsinger et al., 1989, Proc. NatL. Acad. Sci. USA 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the nucleic acid molecules may be conjugated to another molecule, e.g., a peptide, hybridization triggered cross-linking agent, transport agent, hybridization-triggered cleavage agent, etc.

Various other well-known modifications to the nucleic acid molecules can be introduced as a means of increasing intracellular stability and half-life. Possible modifications include, but are not limited to, the addition of flanking sequences of ribonucleotides to the 5′ and/or 3′ ends of the molecule. In some circumstances where increased stability is desired, nucleic acids having modified internucleoside linkages such as 2′-0-methylation may be preferred. Nucleic acids containing modified intemucleoside linkages may be synthesized using reagents and methods that are well known in the art (see, Uhlmann et al., 1990, Chem. Rev. 90:543-584; Schneider et al., 1990, Tetrahedron Lett. 31:335 and references cited therein).

The PTMs of the present invention are preferably modified in such a way as to increase their stability in the cells. Since RNA molecules are sensitive to cleavage by cellular ribonucleases, it may be preferable to use as the competitive inhibitor a chemically modified oligonucleotide (or combination of oligonucleotides) that mimics the action of the RNA binding sequence but is less sensitive to nuclease cleavage. In addition, the synthetic PTMs can be produced as nuclease resistant circular molecules with enhanced stability (Puttaraju et al., 1995, Nucleic Acids Symposium Series No. 33:49-51; Puttaraju et al., 1993, Nucleic Acid Research 21:4253-4258). Other modifications may also be required, for example to enhance binding, to enhance cellular uptake, to improve pharmacology or pharmacokinetics or to improve other pharmaceutically desirable characteristics.

Modifications, which may be made to the structure of the synthetic PTMs include but are not limited to backbone modifications such as use of:

(i) phosphorothioates (X or Y or W or Z=S or any combination of two or more with the remainder as O). e.g. Y=S (Stein, C. A., et al., 1988, Nucleic Acids Res., 16:3209-3221), X=S (Cosstick, R., et al., 1989, Tetrahedron Letters, 30, 4693-4696), Y and Z=S (Brill, W. K.-D., et al., 1989, J Amer. Chem. Soc., 111:2321-2322); (ii) methylphosphonates (e.g. Z=methyl (Miller, P. S., et al., 1980, J Biol. Chem., 255:9659-9665); (iii) phosphoramidates (Z=N-(alkyl)₂ e.g. alkyl methyl, ethyl, butyl) (Z=morpholine or piperazine) (Agrawal, S., et al., 1988, Proc. Natl. Acad. Sci. USA 85:7079-7083) (X or W=NH) (Mag, M., et al., 1988, Nucleic Acids Res., 16:3525-3543); (iv) phosphotriesters (Z=O-alkyl e.g. methyl, ethyl, etc) (Miller, P. S., et al, 1982, Biochemistry, 21:5468-5474); and (v) phosphorus-free linkages (e.g. carbamate, acetamidate, acetate) (Gait, M. J., et al., 1974, J Chem. Soc. Perkin I, 1684-1686; Gait, M. J., et al., 1979, J. Chem. Soc. Perkin I, 1389-1394). See also, Sazani et al., 2001, Nucleic Acids Research 29:3965-3974.

In addition, sugar modifications may be incorporated into the PTMs of the invention. Such modifications include but are not limited to the use of: (i) 2′-ribonucleosides (R=H); (ii) 2′-O-methylated nucleosides (R=OMe) (Sproat, B. S., et al., 1989, Nucleic Acids Res., 17:3373-3386); and (iii) 2′-fluoro-2′-riboucleosides (R=F) (Krug, A., et al., 1989, Nucleosides and Nucleotides, 8:1473-1483).

Further, base modifications that may be made to the PTMs, including but not limited to use of: (i) pyrimidine derivatives substituted in the 5-position (e.g. methyl, bromo, fluoro etc) or replacing a carbonyl group by an amino group (Piccirilli, J. A., et al., 1990, Nature, 343:33-37); (ii) purine derivatives lacking specific nitrogen atoms (e.g. 7-deaza adenine, hypoxanthine) or functionalized in the 8-position (e.g. 8-azido adenine, 8-bromo adenine) (for a review see Jones, A. S., 1979, Int. J Biolog. Macromolecules, 1:194-207).

In addition, the PTMs may be covalently linked to reactive functional groups, such as: (i) psoralens (Miller, P. S., et al., 1988, Nucleic Acids Res., Special Pub. No. 20, 113-114), phenanthrolines (Sun, J-S., et al., 1988, Biochemistry, 27:6039-6045), mustards (Vlassov, V. V., et al, 1988, Gene, 72:313-322) (irreversible cross-linking agents with or without the need for co-reagents); (ii) acridine (intercalating agents) (Helene, C., et al., 1985, Biochimie, 67:777-783); (iii) thiol derivatives (reversible disulphide formation with proteins) (Connolly, B. A., and Newman, P. C., 1989, Nucleic Acids Res., 17:4957-4974); (iv) aldehydes (Schiffs base formation); (v) azido, bromo groups (UV cross-linking); or (vi) ellipticines (photolytic cross-linking) (Perrouault, L., et al., 1990, Nature, 344:358-360).

In an embodiment of the invention, oligonucleotide mimetics in which the sugar and internucleoside linkage, i.e., the backbone of the nucleotide units, are replaced with novel groups can be used. For example, one such oligonucleotide mimetic which has been shown to bind with a higher affinity to DNA and RNA than natural oligonucleotides is referred to as a peptide nucleic acid (PNA) (for review see, Uhlmann, E. 1998, Biol. Chem. 379:1045-52). Thus, PNA may be incorporated into synthetic PTMs to increase their stability and/or binding affinity for the target pre-mRNA.

In another embodiment of the invention synthetic PTMs may covalently linked to lipophilic groups or other reagents capable of improving uptake by cells. For example, the PTM molecules may be covalently linked to: (i) cholesterol (Letsinger, R. L., et al., 1989, Proc. Natl. Acad. Sci. USA, 86:6553-6556); (ii) polyamines (Lemaitre, M., et al., 1987, Proc. Natl. Acad. Sci, USA, 84:648-652); other soluble polymers (e.g. polyethylene glycol) to improve the efficiently with which the PTMs are delivered to a cell. In addition, combinations of the above identified modifications may be utilized to increase the stability and delivery of PTMs into the target cell.

The PTMs of the invention can be used in methods designed to produce a novel chimeric RNA in a target cell. The methods of the present invention comprise delivering to the target cell a PTM RNA molecule, wherein said PTM binds to a pre-mRNA and mediates a trans-splicing reaction resulting in formation of a chimeric RNA comprising a portion of the PTM molecule spliced to a portion of the pre-mRNA.

### 5.2 Synthesis of the Trans-Splicing Molecules

The synthetic PTMs of the invention are typically nucleic acid molecules or derivatives or modified versions thereof, single-stranded or double-stranded. The synthetic PTMs of the invention are preferably RNA molecules composed of ribonucleosides with phosphodiester linkages or modified linkages. The term nucleic acid also specifically includes nucleic acids composed of bases other than the five biologically occurring bases (adenine, guanine, thymine, cytosine and uracil). In addition, the synthetic PTMs of the invention may comprise, DNA/RNA, RNA/protein or DNA/RNA/protein chimeric molecules that are designed to enhance the stability of the PTMs.

The synthetic PTMs of the invention can be prepared by any method known in the art for the synthesis of nucleic acid molecules. For example, the nucleic acids may be chemically synthesized using commercially available reagents and synthesizers by methods that are well known in the art (see, e.g., Gait, 1985, Oligonucleotide Synthesis: A Practical Approach, IRL Press, Oxford, England).

Alternatively, synthetic PTMs can be generated by in vitro transcription of DNA sequences encoding the PTM of interest. Such DNA sequences can be incorporated into a wide variety of vectors downstream from suitable RNA polymerase promoters such as the T7, SP6, or T3 polymerase promoters. Consensus RNA polymerase promoter sequences include the following:

T7: TAATACGACTCACTATAGGGAGA
SP6: ATTTAGGTGACACTATAGAAGNG
T3: AATTAACCCTCACTAAAGGGAGA.

The base in bold is the first base incorporated into RNA during transcription. The underline indicates the minimum sequence required for efficient transcription.

RNAs may be produced in high yield via in vitro transcription using plasmids such as SPS65 and Bluescript (Promega Corporation, Madison, Wis.). In addition, RNA amplification methods such as Q-βamplification can be utilized to produce the PTM of interest.

In addition, the PTMs can be generated by in vivo transcription within a cell. The DNA encoding the PTM of interest may be recombinantly engineered into a variety of host vector systems that also provide for replication of the DNA in large scale and contain the necessary elements for directing high level transcription of the PTM. For example, a vector can be introduced in vivo such that it is taken up by a cell and directs the transcription of the PTM molecule. Such a vector can remain episomal or become chromosomally integrated, as long as it can be transcribed to produce large quantities of the desired RNA. Such vectors can be constructed by recombinant DNA technology methods standard in the art.

Vectors encoding the PTM of interest can be plasmid, viral, or others known in the art, used for replication and expression in mammalian cells. Expression of the sequence encoding the PTM can be regulated by any promoter known in the art to act in mammalian, preferably human cells. Such promoters can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et aL, 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42), the viral CMV promoter, the human chorionic gonadotropin-X promoter (Hollenberg et al., 1994, Mol. Cell. Endocrinology 106:111-119), etc. Any type of plasmid, cosmid, YAC or viral vector can be used to prepare the recombinant DNA construct which can be introduced directly into the tissue site. Alternatively, viral vectors can be used which selectively infect the desired target cell. In addition, the vectors encoding the PTM of interest may be designed to encode a PTM having a nucleotide tag that may be used to efficiently purify the PTM from the cell using affinity chromatography.

A selectable mammalian expression vector system can also be utilized. A number of selection systems can be used, including but not limited to selection for expression of the herpes simplex virus thymidine kinase, hypoxanthine-guanine phosphoribosyltransterase and adenine phosphoribosyl tranferase protein in tk-, hgprt- or aprt- deficient cells, respectively. Also, anti-metabolic resistance can be used as the basis of selection for dihydrofolate tranferase (dhfr), which confers resistance to methotrexate; xanthine-guanine phosphoribosyl transferase (gpt), which confers resistance to mycophenolic acid; neomycin (neo), which confers resistance to aminoglycoside G-418; and hygromycin B phosphotransferase (hygro) which confers resistance to hygromycin. In a preferred embodiment of the invention, the cell culture is transformed at a low ratio of vector to cell such that there will be only a single vector, or a limited number of vectors, present in any one cell. Vectors for use in the practice of the invention include any eukaryotic expression vectors, including but not limited to viral expression vectors such as those derived from the class of retroviruses or adeno-associated viruses.

The PTMs may be purified by any suitable means, as are well known in the art. For example, the PTMs can be purified by gel filtration, affinity or antibody interactions, reverse phase chromatography or gel electrophoresis. Of course, the skilled artisan will recognize that the method of purification will depend in part on the size, charge and shape of the nucleic acid to be purified.

The PTM's of the invention, whether synthesized chemically, in vitro, or in vivo, can be synthesized in the presence of modified or substituted nucleotides to increase stability, uptake or binding of the PTM to a target pre-mRNA. In addition, following synthesis of the PTM, the PTMs may be modified with peptides, chemical agents, antibodies, or nucleic acid molecules, for example, to enhance the physical properties of the PTM molecules. Such modifications are well known to those of skill in the art.

### 5.3 Uses and Administration of Trans-Splicing Molecules

The compositions and methods of the present invention will have a variety of different applications including gene regulation, gene repair, targeted cell death and real time imaging. For example, trans-splicing can be used to introduce a protein with toxic properties into a cell. In addition, synthetic PTMs can be engineered to bind to viral MRNA and destroy the function of the viral MRNA, or alternatively, to destroy any cell expressing the viral mRNA. In yet another embodiment of the invention, synthetic PTMs can be engineered to place a stop codon in a deleterious mRNA transcript thereby decreasing the expression of that transcript.

Targeted trans-splicing, including double-trans-splicing reactions, 3′ exon replacement and/or 5′ exon replacement can be used to repair or correct transcripts that are either truncated or contain mutations. The synthetic PTMs of the invention are designed to trans-splice a targeted transcript upstream or downstream of a specific mutation or upstream of a premature 3′ termination and correct the mutant transcript via a trans-splicing reaction which replaces the portion of the transcript containing the mutation with a functional or therapeutic sequence.

In addition, double trans-splicing reactions may be used for the selective expression of a toxin in tumor cells. For example, synthetic PTMs can be designed to replace the second exon of the human β-chronic gonadotropin-6 (βhCG6) gene transcripts and to deliver an exon encoding the subunit A of diptheria toxin (DT-A). Expression of DT-A in the absence of subunit B should lead to toxicity only in the cells expressing the gene. βhCG6 is a prototypical target for genetic modification by trans-splicing. The sequence and the structure of the βhCG6 gene are completely known and the pattern of splicing has been determined. The βhCG6 gene is highly expressed in many types of solid tumors, including many non-germ line tumors, but the βhCG6 gene is silent in the majority cells in a normal adult. Therefore, the βhCG6 pre-mRNA represents a desirable target for a trans-splicing reaction designed to produce tumor-specific toxicity.

The first exon of βhCG6 pre-mRNA is ideal in that it encodes only five amino acids, including the initiator AUG, which should result in minimal interference with the proper folding of the DT-A toxin while providing the required signals for effective translation of the trans-spliced mRNA. The DT-A exon, which is designed to include a stop codon to prevent chimeric protein formation, will be engineered to trans-splice into the last exon of the βhCG6 gene. The last exon of the βhCG6 gene provides the construct with the appropriate signals to polyadenylate the mRNA and ensure translation.

Cystic fibrosis (CF) is one of the most common fatal genetic disease in humans. Based on both genetic and molecular analyses, the gene associated with cystic fibrosis has been isolated and its protein product deduced (Kerem, B.S. et al., 1989, Science 245:1073-1080; Riordan et al., 1989, Science 245:1066-1073;Rommans, et al., 1989, Science 245:1059-1065). The protein product of the CF associated gene is called the cystic fibrosis transmembrane conductance regulator (CFTR). In a specific embodiment of the invention, a trans-splicing reaction will be used to correct a genetic defect in the DNA sequence encoding the cystic fibrosis transmembrane regulator (CFTR) whereby the DNA sequence encoding the cystic fibrosis trans-membrane regulator protein is expressed and a functional chloride ion channel is produced in the airway epithelial cells of a patient.

Population studies have indicated that the most common cystic fibrosis mutation is a deletion of the three nucleotides in exon 10 that encode phenylalanine at position 508 of the CFTR amino acid sequence. As described in U.S. Pat. No. 6,280,978, a trans-splicing reaction was capable of correcting the deletion at position 508 in the CFTR amino acid sequence. The PTM used for correction of the genetic defect contained a CFTR binding domain complementary to intron 9 sequence, a spacer sequence, a branch point, a polypyrimidine tract, a 3′ splice site and a wild type CFTR BD exon 10 sequence. The successful correction of the mutated DNA encoding CFTR utilizing a trans-splicing reaction supports the general application of PTMs for correction of genetic defects.

The methods and compositions of the invention may also be used to regulate gene expression in plants. For example, trans-splicing may be used to place the expression of any engineered gene under the natural regulation of a chosen target plant gene, thereby regulating the expression of the engineered gene. Trans-splicing may also be used to prevent the expression of engineered genes in non-host plants or to convert an endogenous gene product into a more desirable product.

Various delivery systems are known and can be used to transfer the compositions of the invention into cells, including conjugating PTMs with cationic lipids (Lu,D., et al., 1994, Cancer Gene Ther., 1:245-252) or polycations, DEAE-dextran (Malone, R. W., et al, 1989, Proc. Natl Acad. Sci. USA, 86:6077-6081), poly(L-lysine) (Fisher,K. J. and Wilson, J. M., 1997, Biochem. J., 321:49-58) or dendrimers (Strobel, I., et al., 2000, Gene Ther., 7:2028-2035).

In a specific embodiment of the invention, a composition may be prepared in which the synthetic PTMs are associated with, or impregnated within, a matrix, to form a “matrix-PTM composition” and the matrix-PTM composition is then placed in contact with the cells or tissue expressing the target mRNA. The matrix may become impregnated with the synthetic PTM simply by soaking the matrix in a solution containing the synthetic PTM for a brief period of time of anywhere from about 5 minutes or so, up to and including about an hour. Matrix-PTM compositions are all those in which a synthetic PTM is adsorbed, absorbed, or otherwise maintained in contact with the matrix.

The type of matrix that may be used in the compositions and methods of the invention is virtually limitless, so long as it is a “biocompatible matrix.” This means that the matrix has all the features commonly associated with being “biocompatible,” in that it is in a form that does not produce an adverse, allergic or other untoward reaction when administered to an animal, and it is also suitable for placing in contact with cells or tissue.

Direct in vivo synthetic PTM transfer may be achieved with formulations of synthetic PTMs trapped in liposomes (Ledley et al., 1987); or in proteoliposomes that contain viral envelope receptor proteins (Nicolau et al., 1983) The present invention relates to the synthesis of novel cationic, amphiphilic lipids and their application as synthetic PTM transfer vehicles in vitro and in vivo. A variety of different lipids (diglycerides, steroids) can be synthesized and modifed with variable cationic molecules (amino acids, biogenic amines). Compounds of this kind are, due to their capability of producing complexes with polynucleotides (DNA, RNA, Antisense oligonucleotides, ribozymes, etc.) suitable as vectors for PTMs (transfection) (See, U.S. Pat. No. 6,268,516; Felgner P.L., et al., 1987, Lipofection: a highly efficient, lipid-mediated DNA transfection procedure, Proc. Natl. Acad. Sci. USA 84:7413-7417).

In order to achieve a highly efficient gene transfer both in vitro and in vivo the cationic lipids employed for the generation of liposomes should be non-toxic, fully biodegradable and should not cause an immunoreaction. In addition, the liposomes should form complexes with the synthetic PTMs with high efficacy, protect the synthetic PTM against degradation, and provide high transfection efficiencies. In a preferred embodiment of the invention, liposomes can be engineered in a receptor specific manner. Methods for synthesis of cationic lipids are well known to those of skill in the art.

Receptor-mediated gene transfer may also be used to introduce synthetic PTMs into target cells, both in vitro and in vivo. Such transfer involves linking the synthetic PTM to a polycationic protein (usually poly-L-lysine) containing a covalently attached ligand, which is selected to target a specific receptor on the surface of the cell of interest. The nucleic acid is taken up by the cell and expressed. Cell-specific delivery of a synthetic PTM using a conjugate of a polynucleic acid binding agent (such as polylysine, polyarginine, polyomithine, histone, avidin, or protamine) and a tissue receptor-specific protein ligand may also be achieved using the method of Wu et al. (U.S. Pat. No. 5,166,320).

In yet another embodiment of the invention the “naked” synthetic PTM may be directly injected into the host (Dubenski et al., Proc. Natl. Acad. Sci. USA, 81:7529-33 (1984)). The synthetic PTM may be precipitated using calcium phosphate and injected together with a suitable carrier.

The compositions and methods can be used to treat cancer and other serious viral infections, autoimmune disorders, and other pathological conditions in which the alteration or elimination of a specific cell type would be beneficial. Additionally, the compositions and methods may also be used to provide a gene encoding a functional biologically active molecule to cells of an individual with an inherited genetic disorder where expression of the missing or mutant gene product produces a normal phenotype.

The present invention also provides for pharmaceutical compositions comprising an effective amount of a synthetic PTM and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the synthetic is administered. Examples of suitable pharmaceutical carriers are described in “Remington's Pharmaceutical sciences” by E. W. Martin.

In specific embodiments, pharmaceutical compositions are administered: (1) in diseases or disorders involving an absence or decreased (relative to normal or desired) level of an endogenous protein or function, for example, in hosts where the protein is lacking, genetically defective, biologically inactive or underactive, or under expressed; or (2) in diseases or disorders wherein, in vitro or in vivo, assays indicate the utility of synthetic PTMs that inhibit the function of a particular protein. The activity of the protein encoded for by the chimeric mRNA resulting from the synthetic PTM mediated trans-splicing reaction can be readily detected, e.g., by obtaining a host tissue sample (e.g., from biopsy tissue) and assaying it in vitro for mRNA or protein levels, structure and/or activity of the expressed chimeric mRNA. Many methods standard in the art can be thus employed, including but not limited to immunoassays to detect and/or visualize the protein encoded for by the chimeric mRNA (e.g., Western blot, immunoprecipitation followed by sodium dodecyl sulfate polyacrylamide gel electrophoresis, immunocytochemistry, etc.) and/or hybridization assays to detect formation of chimeric mRNA expression by detecting and/or visualizing the presence of chimeric mRNA (e.g., Northern assays, dot blots, in situ hybridization, and Reverse-Transcription PCR, etc.), etc. Alternatively, direct visualization of a reporter gene either encoded by the synthetic PTM or associated with a PTM may be carried out.

The present invention also provides for pharmaceutical compositions comprising an effective amount of a synthetic PTM or a nucleic acid encoding a synthetic PTM, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical sciences” by E. W. Martin. In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Other control release drug delivery systems, such as nanoparticles, matrices such as controlled-release polymers, hydrogels.

The synthetic PTM will be administered in amounts which are effective to produce the desired effect in the targeted cell. Effective dosages of the synthetic PTMs can be determined through procedures well known to those in the art which address such parameters as biological half-life, bioavailability and toxicity. The amount of the composition of the invention which will be effective will depend on the nature of the disease or disorder being treated, and can be determined by standard clinical techniques. In addition, in vitro assays may optionally be employed to help identify optimal dosage ranges.

The present invention also provides a pharmaceutical pack or kit comprising one or more containers filled with one or more of the ingredients of the pharmaceutical compositions of the invention optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLE

### Production of Trans-Splicing Molecules

The following example demonstrates successful transfer of PTMs into cells and accurate replacement of an internal exon by a double-trans-splicing between a target pre-mRNA and a PTM RNA containing both 3′ and 5′ splice sites leading to production of full length functionally active protein.

### 6.1. Material and Methods

6.1.1. In Vitro Transcription and Purification of RNA

Preparation of Template DNA: Plasmids, pc3.1DSPTM7 and pc3.1DSPTM19 (containing T7 promoter) are described in patent application Serial No. 09/941,492. The plasmids were digested with Stu I restriction enzyme at 37° C. for completion (2-3 hr). The products were extracted once with buffered phenol and once with chloroform or purified using Qiaquick PCR purification kit (Qiagen). The DNA was recovered by ethanol precipitation and was washed twice with 70% ethanol, air dried for 5 min, re-suspended with sterile water and used for in vitro transcription.

Transcription was carried out in 20 μl reaction using either mMESSAGE mMACHINE high yield capped RNA transcription kit for capped RNA or T7-MEGAscript kit for making un-capped RNA by in vitro transcription following manufactures protocol (Ambion) and 1 μg of linearized plasmid DNA template. The reactions were incubated at 37° C. for 2-3 hr and the DNA template was destroyed by adding 1 μl of DNaseI (2U/ml) and continuing the incubation at 37° C. for an additional 15 min. The reactions were terminated by adding formamide gel loading buffer followed by heating for 3 min at 95° C.

In vitro transcribed RNA was purified on a 5-6% denaturing polyacrylamide gel. RNA bands were visualized by UV shadowing. The RNA was eluted from the gel into 0.1% SDS, 10 mM EDTA and recovered by ethanol precipitation, washed twice with 70% ethanol, air dried, re-suspended in sterile water and used for transfections.

### 6.1.2. Synthetic PTM Transfections

The day before transfection, 1×10⁶ 293T cells or double splicing stable cells which express an integrated defective lacZ target pre-mRNA were plated in 60 mm tissue culture plate with 5 ml of DMEM growth medium supplemented with 10% FBS. The cells were incubated at 37° C. in a CO₂ incubator for 12-16 hr or until the cells are ˜60-70% confluent. Before transfection, the cells were washed with 2 ml Opti-MEM I reduced serum medium. The synthetic PTM-Lipid complexes were prepared by adding 1.7 ml of Opti-MEM I into 15 ml tube followed by 8 μl of DMRIE-C transfection reagent (Life Technologies, Bethesda, MD) and mixed briefly. To the above mix, 2.5 and 5.0 μg in vitro transcribed, gel purified RNA was added, vortexed briefly and immediately added to the washed cells. The cells were incubated for 4 hr at 37° C. and then the transfection medium was replaced with complete growth medium (DMEM with 10% FBS). After incubating for an additional 16-24 hr, the plates were rinsed with PBS, cells harvested and total RNA was isolated using MasterPure RNA/DNA purification kit (Epicenter Technologies, Madison, Wis.). Contaminating DNA in the RNA preparation was removed by treating with DNaseI at 37° C. for 30-45 min.

### 6.1.3. Reverse Transcription and Polymerase Chain Reaction

Total cell RNA (2.5 μg) from the transfections was converted to cDNA using the MMLV reverse transcriptase enzyme (Promega) in a 25 μl reaction following the manufacturers protocol with the addition of 50 units RNase Inhibitor (Life Technologies) and 200 ng Lac-6R gene specific primer:

(5′-CTAGGCGGCCGCCTGCTGGTGTTTTGCTTCC).

cDNA synthesis reactions were incubated at 42° C. for 60 min followed by incubation at 95° C. for 5 min. This cDNA template was used for PCR reactions. PCR amplifications were performed using 100 ng primers and 1 μl template (RT reaction) per 50 μl PCR reaction. A typical reaction contained ˜25 ng of cDNA template, 100 ng of primers (common to cis- and trans-spliced products) (KI-1F, 5′-GTTTCGCTAAATACTGGCAGG and, Lac-6R, 5′-CTAGGCGGCCGCCTGCTGGTGTTTTGCTTCC) 1×REDTaq PCR buffer (10 mM Tris-HCl, pH 8.3, 50 mM KCl, 1.1 mM MgCl₂ and 0.1% gelatin), 200 μM dNTPs and 1.5 units of REDTaq DNA polymerase (Sigma, Saint Louis, Mo.). PCR reactions were performed with an initial pre-heating at 94° C. for 2 min 30 sec followed by 20 cycles of 94° C. for 30 sec (denaturation), 60° C. for 36 sec (annealing) and 72° C. for 1 min (extension) followed by a final extension at 72° C. for 7 min. The PCR products were then digested with Sph I and Dde I restriction endonucleases, which specifically cleaves cis-spliced product. Trans-spliced product was isolated using Lac-21 (has biotin at the 5′ end) as a hybridization probe. The purified trans-spliced product was subjected to a 2nd round of nested PCR using primers KI-2F (5′-CTGGCAGGCGTTTCGTCAG) and Lac-6R. Authenticity of the trans-spliced product was further confirmed by diagnostic digestion with Pvu I restriction enzyme which specifically cleaves the trans-spliced product.

### 6.1.4. β-Galactosidase Assay

Total cellular protein was isolated by freeze thaw method and assayed for β-galactosidase activity using a β-gal assay kit (Invitrogen, Carlsbad, Calif.). Protein concentration was measured by the dye-binding assay using Bio-Rad protein assay reagents (BIO-RAD, Hercules, Calif.).

### 6.1.2. Results

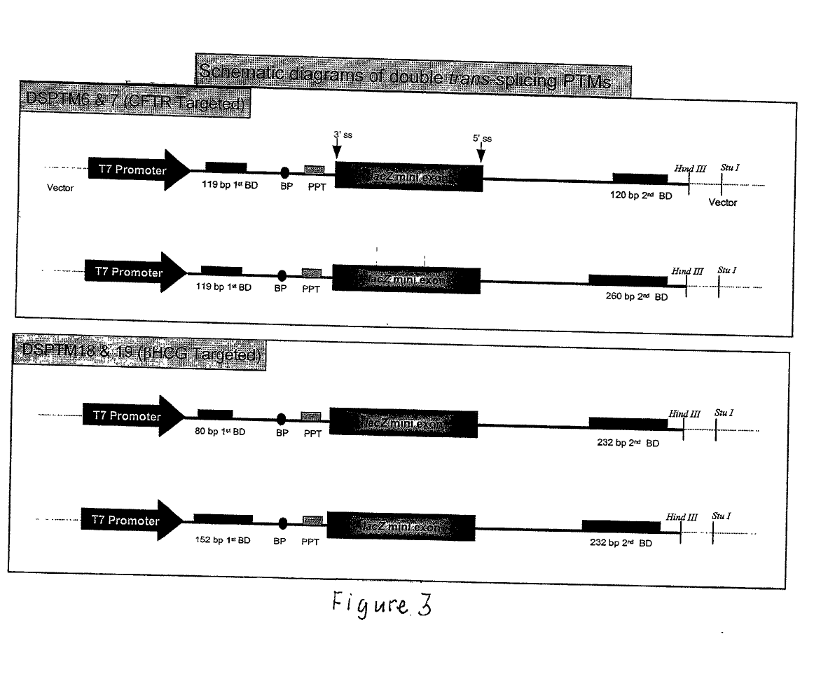

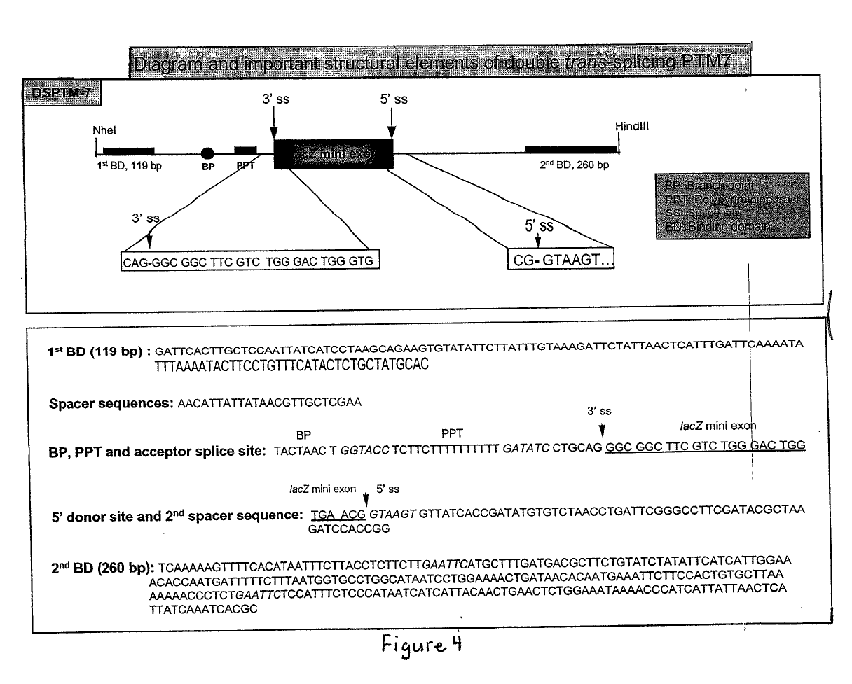

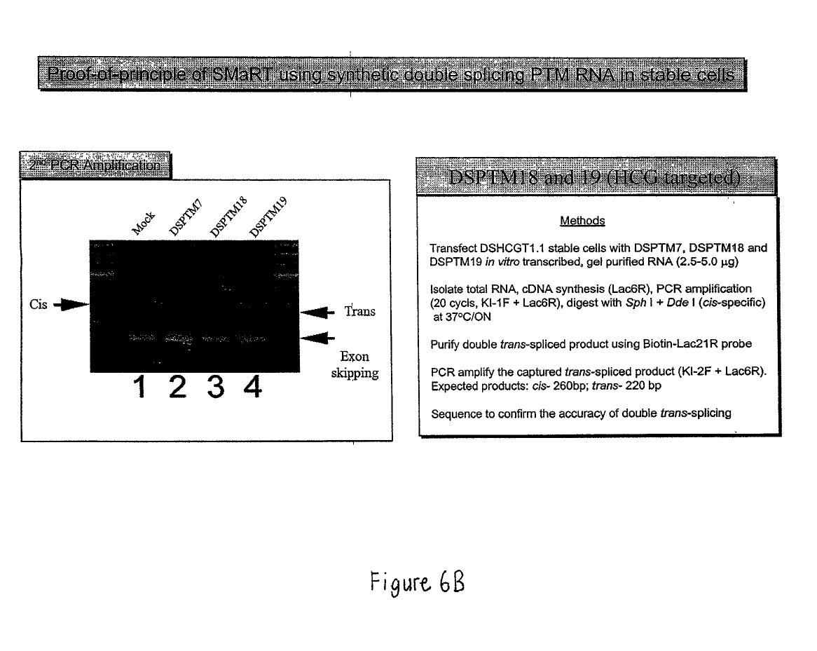

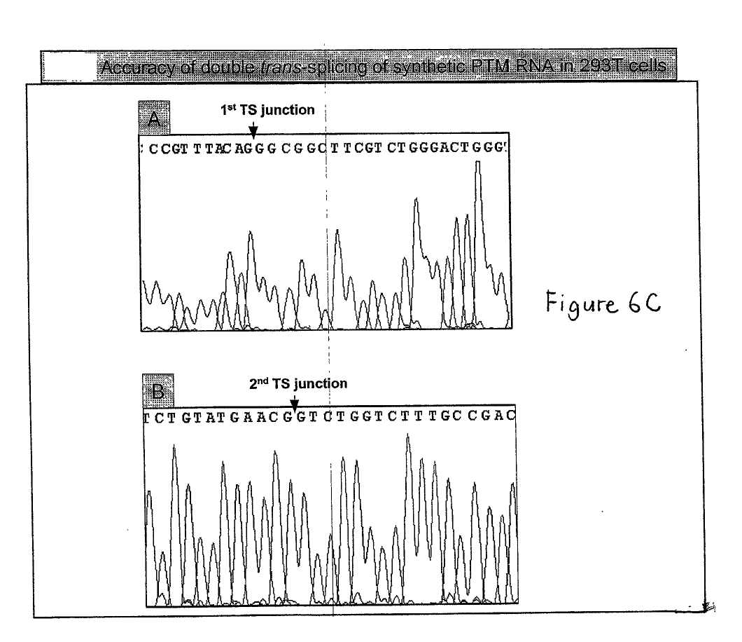

Using in vitro synthesized PTM RNA as genetic material, the results described herein demonstrated the accurate trans-splicing of double splicing exogenously synthesized PTM RNAs (DSPTM7, CFTR targeted; DSPTM19, βHCG targeted) into pre-mRNA target (FIG. 2) both in co-transfection assays (293T cells) as well as in cells that express double splicing lacZ pre-mRNA target from an integrated genomic locus. For this purpose, DSPTM6, DSPTM7, DSPTM18 and DSPTM19 (capped and uncapped) (FIG. 3) RNAs were exogenously synthesized using bacteriphage T7 RNA polymerase in vitro, gel purified and used for transfections. 48 hrs post-transfection, total cellular RNA was isolated and analyzed by RT-PCR (as described above). As shown in FIG. 6, both DSPTM6 and DSPTM7 produced the expected trans-spliced 220 bp RT-PCR product in 293T cells (upper panel). The authenticity of this product was confirmed by diagnostic digestion using Sph I, which cuts the cis-spliced product specifically (lower panel, lanes 1 and 2) and Pvu I that cuts the trans-spliced product specifically (lower panel, lanes 4 and 5). To confirm that trans-splicing between DSPTM7 and DSCFT1.6 pre-mRNA is precise, RT-PCR amplified product was excised, re-amplified using KI-2F and Lac6R primers and sequenced directly using KI-2F or Lac-6R primers. As shown in FIG. 6C, trans-splicing occurred exactly at the predicted splice sites, confirming the precise internal exon substitution by the double trans-splicing events.

Trans-splicing efficiency and specificity of DSPTM18, DSPTM19 and DSPTM7 were tested in stable cells that express double splicing HCG target pre-mRNA endogenously. RT-PCR analyses of the total RNA that were transfected with DSPTM18 and DSPTMl9 produced the expected 220 bp trans-spliced product (FIG. 6B, lanes 3 and 4). No trans-spliced product was detected in cells that were mock transfected or transfected with DSPTM7 that is targeted to CFTR target pre-mRNA (lanes 1 and 2).

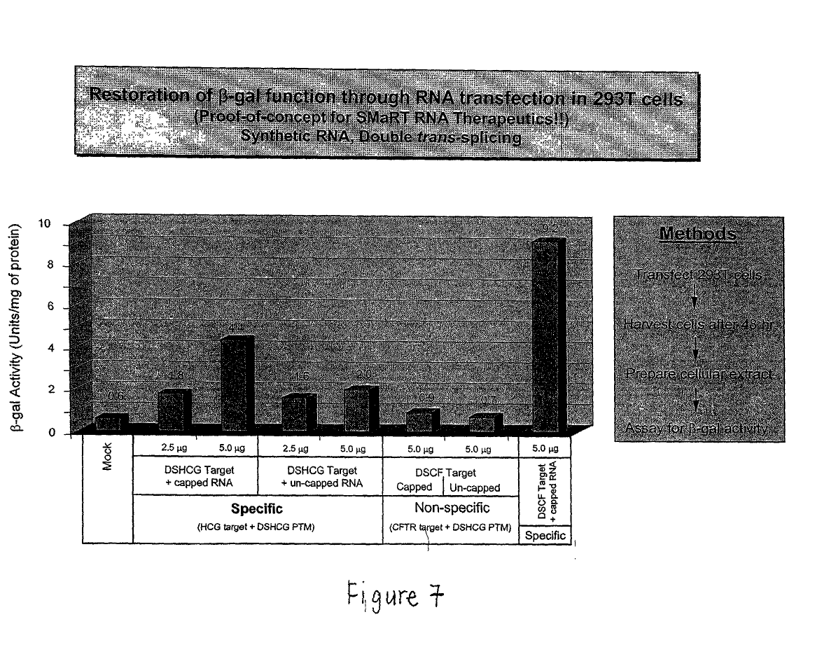

The efficiency and specificity of double trans-splicing mediated restoration of protein function was further confirmed at the protein level by assaying for β-gal activity. The results are summarized in FIG. 7. Co-transfection of specific target with either capped or uncapped in vitro transcribed PTM RNA resulted in the repair and restoration of β-gal function in both CFTR (15-fold above the background) and βHCG (7-fold higher) models. The trans-splicing efficiency of capped PTM RNA was almost 2-fold higher than the uncapped PTM RNA, suggesting that the capped RNA may be more stable. The specificity of double trans-splicing was evaluated by co-transfecting synthetic DSPTM19 (capped and uncapped) PTM RNA along with a non-specific target (DSCFT1.6). The level of β-gal activity in cells that were transfected with non-specific target was almost identical to that of mock transfection suggesting a high level of specificity for the double trans-splicing reaction.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

We claim:

1. A method of producing a chimeric RNA molecule in a cell comprising: contacting a cell with a nucleic acid molecule recognized by nuclear splicing components wherein said nucleic acid molecule comprises:
a) one or more target binding domains that target binding of the nucleic acid molecule to a pre-mRNA expressed within the cell;
b) a 3′ splice region comprising a branch point, a pyrimidine tract and a 3′ splice acceptor site; and
c) a nucleotide sequence to be trans-spliced to the target pre-mRNA;
wherein said nucleic acid molecule is recognized by nuclear splicing components within the cell.

2. A method of producing a chimeric RNA molecule in a cell comprising: contacting a cell with a nucleic acid molecule recognized by nuclear splicing components wherein said nucleic acid molecule comprises:
a) one or more target binding domains that target binding of the nucleic acid molecule to a pre-mRNA expressed within the cell;
b) a 3′ splice acceptor site; and
c) a nucleotide sequence to be trans-spliced to the target pre-mRNA;
wherein said nucleic acid molecule is recognized by nuclear splicing components within the cell.

3. A method of producing a chimeric RNA molecule in a cell comprising contacting a cell with a nucleic acid molecule recognized by nuclear splicing components wherein said nucleic acid molecule comprises:
a) one or more target binding domains that target binding of the nucleic acid molecule to a pre-mRNA expressed within the cell;
b) a 5′ splice site; and
c) a nucleotide sequence to be trans-spliced to the target pre-mRNA;
wherein said nucleic acid molecule is recognized by nuclear splicing components within the cell.

4. The method of claim 1 wherein the nucleic acid molecule further comprises a 5′ donor site.

5. The method of claim 1, 2, 3 or 4 wherein said nucleic acid molecule further comprising a spacer region that separates the 3′ splice region from the target binding domain.

6. The method of claim 1, 2, 3, or 4 wherein said nucleic acid molecule further comprising a safety sequence comprising one or more complementary sequences that bind to one or both sides of the 3′ splice site.

7. The method of claim 1, 2, 3, or 4 wherein binding of said nucleic acid molecule binds to the target pre-mRNA is mediated by complementary, triple helix formation, or protein-nucleic acid interaction.

8. The method of claim 5 wherein binding of the nucleic acid molecule to the target pre-mRNA is mediated by complementary, triple helix formation, or protein-nucleic acid interaction.

9. The method of claim 6 wherein the binding of the nucleic acid molecule to the target pre-mRNA is mediated by complementary, triple helix formation, or protein-nucleic acid interaction.

10. The method of claim 1, 2, 3 or 4 wherein the nucleotide to be trans-spliced to the target pre-mRNA encodes a translatable polypeptide.

11. The method of claim 5 wherein the nucleotide to be trans-spliced to the target pre-mRNA encodes a translatable polypeptide.

12. The method of claim 6 wherein the nucleotide to be trans-spliced to the target pre-mRNA encodes a translatable polypeptide.

13. The method of claim 1, 2, 3 or 4 wherein the nucleotide sequence to be trans-spliced to the target pre-mRNA contains a nonsense mutation.

14. The method of claim 5 wherein the nucleotide sequence to be trans-spliced to the target pre-mRNA contains a nonsense mutation.

15. The method of claim 6 wherein the nucleotide sequence to be trans-spliced to the target pre-mRNA contains a nonsense mutation.

16. A method of producing a chimeric RNA molecule in a cell comprising: contacting a cell with a nucleic acid molecule recognized by nuclear splicing components wherein said nucleic acid molecule comprises:
a) one or more target binding domains that target binding of the nucleic acid molecule to a pre-mRNA expressed within the cell;
b) a 5′ donor site;
c) 3′ splice acceptor site; and
c) a nucleotide sequence to be trans-spliced to the target pre-mRNA;
wherein said nucleic acid molecule is recognized by nuclear splicing components within the cell.

17. The method of claim 16 wherein the nucleic acid molecule further comprises a spacer region that separates the 3′ splice region from the target binding domain.

18. The method of claim 16 further comprising a safety sequence comprising one or more complementary sequences that bind one of both sides of the 3′ splice site.
